(19) United States
(12) Patent Application Publication
(10) Pub. No.: US 2008/0269313 A1
(43) Pub. Date: Oct. 30, 2008

(54) Prolyl Oligopeptidase Inhibitors Ameliorating Recovery From Brain Trauma

(75) Inventors: Jouni Sirvio, Kuopio (FI); Tarja Lehtimaki, Sauvo (FI)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: ORION CORPORATION, Espoo (FI)

(21) Appl. No.: 10/563,362

(22) PCT Filed: Jul. 2, 2004
(86) PCT No.: PCT/FI2004/000423

§ 371 Date: May 19, 2006

(30) Foreign Application Priority Data
Jul. 4, 2003 (FI) 20031018

(51) Int. Cl. A61K 31/4025; A61P 25/00

(52) U.S. Cl. 514/422

(57) ABSTRACT

The present invention relates to a method for the treatment of sensorimotor dysfunctions caused by brain trauma with an inhibitor of prolyl oligopeptidase or a pharmaceutically acceptable ester or salt thereof.

### FIELD OF THE INVENTION

The present invention relates to a method for the treatment of sensorimotor dysfunctions caused by brain trauma. More specifically, in such a method a therapeutically effective amount of a prolyl oligopeptidase (POP) inhibitor or its pharmaceutically acceptable ester or salt is administered to a mammal in need thereof.

### BACKGROUND OF THE INVENTION

Patients with cerebral infarction, caused by the lack of supply for blood flow to certain brain areas (‘ischemia’), exhibit sensori-motor disturbances due to a damage of brain tissue supporting those functions (‘stroke’). The duration of sensorimotor disturbances may continue after the acute phase of cerebral infarction. Other causes of stroke than cerebral infarction are primary intracerebral haemorrhage and subarachnoid haemorrhage.

There is no effective treatment available at present to prevent or restrict acute development of cerebral infarction. Most stroke patients, however, exhibit some degree of recovery in their sensorimotor functions, such as the use of hand and fingers as well as in the gait and walking, after initial ischemic insult.

There is growing amount of evidence that the recovery process can be enhanced by physical therapy. The most recent finding is that this recovery process can be more effective by combining physical therapy and drug treatment. The latter opportunity is clinically appealing, since it may be possible to enhance functional recovery even when treatment is initiated days or weeks after cerebral infarction (Feeney D M, Mechanisms of noradrenergic modulation of physical therapy: Effects on functional recovery after cortical injury. In: Goldstein, L. B. (Ed.), Advances in Pharmacotherapy for Recovery After Stroke (1998) pp. 35-78.

Neuroactive peptides act as neurotransmitters or neuromodulators throughout the central nervous system. Their duration of action once released from nerve terminals is limited by the action of various neuropeptidases inactivating them. Prolyl endopeptidase, known also as prolyl oligopeptidase (POP, EC 3.4.21.26), plays a pivotal role in the breakdown of several proline-containing neuropeptides. Subsequently, the prolyl oligopeptidase inhibitor increases the levels of those neuropeptides in the brain (Toide, K., et al., Behavioural Brain Research 83 (1997) 147-151).

This inhibition of the catabolism of neuropeptides can have functional consequences for brain operations. Some POP inhibitors, such as JTP-4819, are known to have cognition improving effects in the experimental models of senile dementia (Toide, K., et al., Behavioural Brain Research 83 (1997) 147-151).

Until now, the investigations on drug treatment on the recovery from cerebral infarction have focussed on monoaminergic transmitters (Goldstein L B, Stroke 21: 139-142, 1990). The present invention describes the positive effects of JTP-4819, a POP inhibitor, on the recovery of limb use after the focal forebrain ischemia. The recovery was assessed using a limb placing task and was shown to be comparable of that with adrenergic treatment (Jolkkonen, J., et al., Eur. J Pharmacol. 400 (2000) 211-219).

### SUMMARY OF THE INVENTION

The object of the present invention is to provide a new method for the treatment of sensorimotor dysfunctions caused by brain trauma, more specifically for facilitating the recovery and/or enhancing the result of recovery in said sensorimotor dysfunctions. The invention describes how a prolyl oligopeptidase inhibitor enhances the recovery in the use of limbs and especially when the reduced use of limbs is caused by a focal forebrain ischemia.

Additional objects and advantages of the invention will be set forth in part in the description, which follows, and in part will be obvious from the description, or may be learned by practice of the invention. The objects and advantages of the invention will be realized and attained by means of the elements and combinations particularly pointed out in the appended claims.

It is to be understood that the foregoing general description and the following detailed description are exemplary and explanatory only and are not restrictive of the invention as claimed.

### BRIEF DESCRIPTION OF THE DRAWINGS

FIG. 1 shows the effect of JTP-4819 on the recovery from focal forebrain brain ischemia in the limb-placing test in rats. Vehicle treated ischemic rats (‘control’) showed a marked reduction in the limb placing score at day 2 (two days after the surgery for a focal, unilateral ischemia) as compared to vehicle treated sham operated rats (‘sham’) who showed a maximal score of this test. Thereafter there was a gradual, partial recovery among vehicle treated ischemic rats as shown by their increased scores. JTP-4819 was given to the rats at two dose levels (0.9 or 9.0 μmol/kg intraperitoneally). JTP-4819, especially the lower dose of the drug, augmented recovery as shown by increased score as compared to vehicle treated rats on day 5-17. Data are expressed as mean scores (SEM) of groups on a limb-placing task. *p<0.05 as compared to vehicle.

### DETAILED DESCRIPTION OF THE INVENTION

The present invention relates to a novel therapeutic approach to facilitate the recovery process following brain trauma in mammals, including human and animals.

Applicants have surprisingly discovered that the daily, systemic administration of JTP-4819, which is the POP inhibitor, enhanced the recovery in the use of limbs in rats with the unilateral focal forebrain ischemia when the drug treatment was started after ischemia. Accordingly, the present findings suggest that the POP inhibitors and their pharmacologically acceptable esters or salts, can be used for the enhancing the rehabilitation from brain trauma, particularly for the facilitating the recovery and/or enhancing the result of recovery in sensorimotor dysfunctions caused by brain trauma. Such dysfunctions include, but are not limited to, reduced sensation of the limbs, an/or disability, handicap or impairment to move the limbs, and/or orofacial muscles. Further, said brain trauma include, but are not limited to, cerebral infarction, primary intracerebral haemorrhage, subarachnoid haemorrhage, cerebral hypoxia, or traumatic brain injury due to e.g. accidents.

The mechanisms underlying recovery after brain trauma are not precisely known. The proposed mechanisms include functional reorganization of brain connections and enhanced function of remaining relevant connections.

Furthermore, the present invention relates the use of a prolyl oligopeptidase inhibitor, or its pharmaceutically acceptable ester or salt thereof, in the manufacture of a pharmaceutical for the treatment of sensorimotor dysfunctions caused by brain trauma, particularly for facilitating the recovery and/or enhancing the result of recovery in said sensorimotor dysfunctions in a mammal.

To achieve optimal results, the treatment with a POP inhibitor is possibly started at few days to several weeks after the brain trauma. The treatment of patient with a POP inhibitor is expected to be optimal if it is combined with physical therapy considered to be relevant for a patient according to her/his condition. The treatment with a POP inhibitor continues as far as the condition of a patient is improving. This improvement can be verified by a neurologist or therapist e.g. by using Fugl-Meyer-Motor Scale (Fugl-Mayer et al. Scand J Rehabil Med 7: 13-31, 1975) or equivalent examination.

The benefit of treatment with POP inhibitor comes from the fact the improvement in the recovery is faster than without this drug treatment, and a patient can be moved from a hospital or rehabilitation center earlier to her/his home ward.

The precise amount of the drug to be administered to a mammal according to the present invention is dependent on numerous factors known to one skilled in the art, such as, the compound to be administered, the general condition of the patient, the condition to be treated, the desired duration of the treatment, the type of mammal, the method and route of administration etc. For example, for the POP inhibitor, the usual daily dosage will be from 1 to 1000 mg, possibly from 1 to 100 mg, divided in 1 to 4 individual doses.

For the purpose of the invention the POP inhibitor or its pharmaceutically acceptable ester or salt can be administered by various routes. Typical routes of administration include, but are not limited to, oral, transdermal, transmucosal, and parenteral routes. One skilled in the art would recognize the dosage forms suitable in the method of the present invention.

For the purposes of the invention the term “treatment” means treatment in order to remedy or alleviate the symptoms of the disorder or condition, and treatment in order to prevent the development or the exacerbation of the symptoms.

For the purpose of the invention the term “sensorimotor dysfunction” means disability, impairment or handicap in the movements of the limbs and/or sensation of the limbs or movements of orofacial muscles.

For the purpose of the invention the term “recovery” means gradual improvement of dysfunction.

The compounds having prolyl oligopeptidase inhibitory activity in the present invention includes any compound having inhibitory activity on an enzyme called prolyl oligopeptidase. Accordingly, the prolyl oligopeptidase inhibitors used for the purpose of the invention include, without limitation, compounds described, for example, in U.S. Pat. No. 6,121,311, WO 03/004468 A1, De Nanteuil, G., et al., Drugs of the Future 23(2) (1998) 167-179 and WO 91/18891,

Furthermore, the use of a prolyl oligopeptidase inhibitor in combination with other medication, that is used in conditions in which brain trauma may appear, would be therapeutically beneficial by providing either an effective treatment to patient resistant to the said conventional therapeutic agents alone, or by providing a synergistic action with the said conventional therapeutic agents. Such other medication include, but is not limited to, psychopharmaceutical drugs (like antidepressants, e.g. alpha2 antagonist), drugs used for thrombolytic therapy (like aspirin) or antihypertension drugs, which drug does not need to have prolyl oligopeptidase inhibitory activity.

The POP inhibitor and the second compound should possibly be administered to the patient during the same period of treatment. The most possibly, the POP inhibitor and the second compound should be administered simultaneously. According to a particularly preferable embodiment, these compounds are administered from the same dosage form.

Such a combination therapy will allow the use of smaller doses of the said compounds and thereby substantially reduce their drug and mechanism specific adverse effects.

The invention will be further clarified by the following example, which is intended to be purely exemplary of the invention.

### EXAMPLE

The effects of JTP-4819 on the recovery in the use of limbs assessed by the limb placing task in male rats with the focal forebrain ischemia.

### Animals

The experiment was carried out on male Spraque-Dawley rats (about 200 g), bred in B&K, Sweden. The rats were allowed to acclimatized for one week before the start of teaching of behavioural testing and subsequent operation (Table 1). During acclimatization period, the rats were housed in solid bottom Macrolon type III cages with stainless steel mesh lids (at maximum five rats in each cage). The rats had free access to standard certified pelleted food (RM1 Maintenance Expanded SQC; Special Diet Services, Essex, UK) and water. After operation, the rats were housed singly in a Macrolon cage. Some food pellets were put into a cage in order to enable to their feeding. On the second post-operative day and thereafter, the rats were housed (maximum 10 rats in one cage) in the enriched environment (Enriched rat cage system, 03-RS-R, Scanbur A/S, Lellinge, Denmark) where the floor area of one cage was 4250 cm². The rats had a shelter, resting shelf, ladders and wooden toys in this kind of cage. Tap water and pelleted food were freely available.

In the vivarium, ambient temperature was 22±1 C.°, and a 12:12 h light/dark cycle was maintained with lights on at 6 A.M. All experiments were carried out between 7 A.M. and 5 P.M. The animal care was performed in accordance with International Council for Laboratory Animal Science (ICLAS) guidelines.

### Ischemia Operation

Anesthesia was initiated with 4-5% halothane (30% oxygen, 65% nitrous oxide) and maintained throughout the operation with 0.5-1.5% halothane delivered via a nose cone. Body temperature was maintained at 37.0±0.5° C. using a thermoregulatory heating unit with homeothermic blanket (HB 101/2 RS, Letica Scientific Instruments). Heparin (40 IU, i.v.) was given just before the operation to prevent possible complications caused by blood coagulation. The right common carotid artery was exposed through a midline ventral cervical incision, and carefully separated from the adjacent sympathetic nerves under a stereomicroscope. The common carotid artery and internal carotid artery were then clamped with microvascular clips. The external carotid artery was ligated distally with a nylon suture and the artery was cut for induction of suture. A heparinized nylon suture (Ø0.28 mm, rounded tip) was introduced into the stump of the external carotid artery, the internal carotid artery clip was removed, and the suture was advanced into the internal carotid artery until it passed beyond the origin of the middle cerebral artery. After occlusion for 90 min, the suture was removed to allow reperfusion in the middle cerebral artery territory. The stump of the external carotid artery was electrocoagulated and the common carotid artery clip was removed. The cervical incision was closed with silk sutures after the application of 2% lidocain gel (Xylocaln® 2%, Astra Finland). In sham-operated rats, the right common carotid artery was exposed and the external carotid artery was electrocoagulated without introducing the filament into the internal carotid artery. Animals were given 0.9% NaCl (i.p.) during the first-post-operative days (days 0 and 1). They were housed singly in a Macrolon cage.

### Drug Treatment

JTP-4819 was dissolved in sterile saline. The drug was injected 1 ml/kg intraperitoneally (i.p.). The drug was administered 30-60 (preferably 45) minutes before testing on days 3-14. On the testing day 2, the drug was injected after the rats were tested in a limb placing test and assigned to groups. On days 3-14, the rats were treated even if they were not tested in a limb placing test Table 1). In those days (6, 8, 10, 12, 13), the rats were injected in the afternoon, about 24 hours after the preceding injection. The rats were weighed on the day 2, 4, 6, 11 and 17 before injections were done. The schedule of the experiment was randomised.

### Groups

On the second day after operation, behavioral deficits of rats were assessed by using a limb-placing test (De Ryck et al., 1989). Animals with a score more than 6 were excluded and the rest of the animals were assigned to following groups:
Sham-operated rats treated with saline (s.c.) and saline (i.p.) (n=4)
Ischemic rats treated with saline (i.p.) (n=8)
Ischemic rats treated with JTP-4819 9 μmol/kg (i.p.) (n=8)
Ischemic rats treated with JTP-4819 1 μmol/kg (i.p.) (n=8)

### Behavioural Test

The limb-placing test was used to assess functional recovery after operation. This test is a modified version of a test described by De Ryck et al. (1989), which assesses the sensorimotor integration of fore- and hind limbs responses to tactile and proprioceptive stimulation. The test had seven limb placing tasks, which were scored: 2 points, the rat performed normally; 1 point, the rat performed with a delay (>2 sec) and/or incompletely and 0 points, the rat did not perform normally. The both sides of the body were tested. In the first task the rat is hanged 10 cm over the table. Normal rats stretched both forelimbs towards the table. On the second task the rat was towards the table holding its forelimbs on it. The forelimb was gently pulled down and the retrieval and placing was checked. Normal rats replaced the limbs to the table. The third task was the same as the second, except that the rat was not able to see the table or contact it by vibrissae by keeping its head upward in 45° angle. The rats were next placed along the table edge to check the lateral placing of the fore- (the fourth task) and hind limbs (the fifth task). In the sixth task the rat were placed again towards the table the hind limbs just over the table edge. The hind limbs were pulled down and gently stimulated by pushing towards the side of the table. In the seventh task the forelimbs of the rat were on the edge of the table and the rat was gently pushed from behind toward the edge. Injured rats could not keep their grip and the injured limb slipped off the edge.

TABLE 1
The study protocol.

| Study day | | Limb placing test |
|---|---|---|
| −4-−1 | | Teaching (2x) |
| 0 | Operation | |
| 2-21 | Enriched environment | |
| 2-14 | Drug/Vehicle treatment | |
| | (begins after baseline | |
| | treatment) | |
| 2 | Weighting | X (Baseline) |
| 3 | | X |
| 4 | Weighting | X |
| 5 | | X |
| 6 | Weighting | |
| 7 | | X |
| 8 | | |
| 9 | | X |
| 10 | | |
| 11 | Weighting | X |
| 12 | | |
| 13 | | |
| 14 | | X |
| 17 | Weighting | X |
| 21 | | X |

### Statistical Analysis

The differences in behavioral scores between experimental groups in the limb placing test were analyzed by Mann-Whitney U-test. The overall group effect on each day was analyzed by Kruskal-Wallis nonparametric analysis of variance. SPSS (v.10 statistical package was used for analysis.

### Results

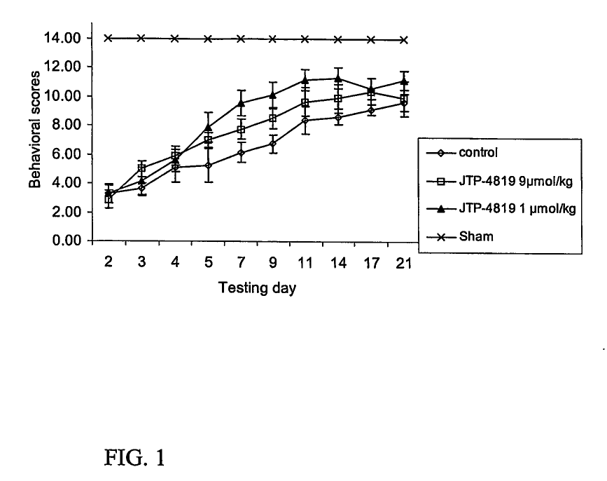

The scoring of the limb-placing test was used to assign rats to comparable groups. The results of the limb-placing test are shown in FIG. 1. When differences in limb-placing tests were assessed between groups, there was a significant difference (p<0.05) between the JTP-4819 (1 μmol/kg) and control groups after ischemia from day 5 to day 17, and JTP-4819 (9 μmol/kg) treatment on day 3 and 9. At a lower dose of JTP-4819, amelioration effect also was seen after the discontinuation of drug administration. The difference in performing a limb-placing test between the ischemic and sham-operated groups remained through the whole testing period although the ischemic groups recovered function of time (FIG. 1.).

Those skilled in the art will appreciate that the embodiments described in this application could be modified without departing from the inventive concept. Those skilled in the art also understand that the invention is not limited to the particular disclosed embodiments, but is intended to also cover modifications to the embodiments that are within the spirit and scope of the invention.

1. A method for treating a sensorimotor dysfunction caused by a brain trauma which comprises administering to a mammal in need of the treatment a therapeutically effective amount of a prolyl oligopeptidase inhibitor, or a pharmaceutically acceptable ester or salt thereof.

2. The method according to claim 1, wherein the brain trauma is brain infarction.

3. The method according to claim 1, wherein the brain trauma is primary intracerebral haemorrhage.

4. The method according to claim 1, wherein the brain trauma is subarachnoid haemorrhage.

5. The method according to claim 1, wherein the brain trauma is brain hypoxia.

6. The method according to claim 1, wherein the brain trauma is traumatic brain injury.

7. The method according to claim 1, wherein the sensorimotor dysfunction is selected from reduced ability to move the limbs and reduced sensation of the limbs.

8. The method according to claim 1, wherein the sensorimotor dysfunction is reduced function of orofacial muscles.

9. The method according to claim 1, wherein the mammal is a human.

10. The method according to claim 1, wherein the prolyl oligopeptidase inhibitor is JTP-4819.
